(19) United States
(12) Patent Application Publication
(10) Pub. No.: US 2005/0074474 A1
(43) Pub. Date: Apr. 7, 2005

(54) Topical composition

(75) Inventor: Takashi Sako, Kobe (JP)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 10/957,565

(22) Filed: Oct. 1, 2004

Related U.S. Application Data
(60) Provisional application No. 60/508,387, filed on Oct. 3, 2003.
(60) Provisional application No. 60/550,105, filed on Mar. 4, 2004.

(51) Int. Cl. A61K 7/42; A61K 7/40

(52) U.S. Cl. 424/401; 424/59

(57) ABSTRACT

Disclosed is a topical composition comprising: (1) a porous spherical disintegrative silica impregnated with a water-insoluble skin benefit agent, wherein: (a) the porous spherical disintegrative silica has an average volume particle size of from about 3 μm to about 20 μm, a maximum particle size of no more than about 50 μm, and a pore volume of from about 1.5 cm³/g to about 3.0 cm³/g; and provides a certain dynamic viscoelasticity when sheared; (b) the water-insoluble skin benefit agent having a solubility in water at less than about 0.1 g/l at 25° C. and having a molecular weight of no more than about 5,000, selected from the group consisting of liquid water-insoluble skin benefit agents, solid water-insoluble skin benefit agents which dissolve in liquid water-insoluble skin benefit agents, solid water-insoluble skin benefit agents which dissolve in emollients and/or volatile solvents, and mixtures thereof; and (2) a suitable carrier.

### CROSS REFERENCE TO RELATED APPLICATIONS

This application claims the benefit of U.S. Provisional Application No. 60/508,387, filed on Oct. 3, 2003 and No. 60/550,105, filed on Mar. 4, 2004.

### FIELD OF THE INVENTION

The present invention relates to a topical composition that comprises a porous spherical disintegrative silica component impregnated with a water-insoluble skin benefit agent. The porous spherical disintegrative silica component is capable of delivering the water-insoluble skin benefit agent to the skin which may otherwise affect physical property or performance of the composition when directly included. The porous spherical disintegrative silica is readily disintegrated upon spreading on the skin. The present invention further relates to various compositions comprising the impregnated porous spherical disintegrative silica.

### BACKGROUND OF THE INVENTION

Lipophilic and water-insoluble components provide important characteristics for topical compositions. Water-insoluble components themselves are emollients or lubricants which protect the skin and allow the product to be applied on the skin. The selection of the type and amount of water-insoluble components are key for determining the overall feel of the product to the skin. Water-insoluble components also act as a carrier for delivering functional components such as pigments and active agents. Further, in solid cosmetic products, water-insoluble components such as waxes and oils act as binders for maintaining the shape and hardness of the product.

Recently, the general consumer has become to seek certain function for topical composition products that can be applied to their daily skin care regimen. Such function may relate to skin hyperpigmentation (age spots, freckles, blotches, darkening, uneven tone, and the like), wrinkling, aging, environmental damage, or protection from environmental damage. One effective way to provide such function is to incorporate skin benefit agents at an effective level in the product. Incorporation of such skin benefit agents must be balanced with the other characteristics of the product, including skin feel, stability, and aesthetics. Namely, when the skin benefit agent is a water-insoluble one, other water-insoluble components are replaced or adjusted with the water-insoluble skin benefit agent for providing the same or similar characteristic. Unfortunately, water-insoluble skin benefit agents tend to provide unfavorable skin feel, and/or interfere with desirable product physical properties of the product. Any of such causes may result in a poor performing, or even unstable product.

Impregnation and encapsulation of ingredients in solid particles are known in the art, such as in Japanese laid-open patent publications 53-38635, 7-330541, 8-165219, and 8-301723. The solid particle selected for such purpose has low reactivity with the ingredients to impregnate/encapsulate, and protects the ingredient from interacting with the product. While these impregnated/encapsulated solid particles may be effective in protecting the incorporated ingredient from interacting with the product, the incorporated ingredient may not be fully utilized on the skin, as being entrapped in the impregnated/encapsulated solid particles.

Disintegrative pigments are known in the art, such as in Japanese laid-open patent publications 3-181410, 5-201830, 2001-158717, 2003-137760 and 2001-322909. These disintegrative pigments disintegrate into finer particles upon the stress applied when spreading the product on the skin, and provide a favorable feel to the skin. Incorporation of water-soluble ingredients in these disintegrative pigments is taught.

Based on the foregoing, there is a need for a topical composition which provides safe and effective skin care treatment benefit via delivering water-insoluble skin benefit agents in a stable manner. There is also a need to incorporate water-insoluble skin benefit agents in a topical composition while providing improved skin feel. There is yet a need to formulate such water-insoluble skin benefit agents in various composition forms in a stable matter. There is yet a need to provide such compositions in an economical manner.

None of the existing art provides all of the advantages and benefits of the present invention.

### SUMMARY OF THE INVENTION

The present invention is directed to a topical composition comprising:

(1) a porous spherical disintegrative silica impregnated with a water-insoluble skin benefit agent, wherein:

(a) the porous spherical disintegrative silica has an average volume particle size of from about 3 μm to about 20 μm, a maximum particle size of no more than about 50 μm, and a pore volume of from about 1.5 cm³/g to about 3.0 cm³/g; wherein when a paste is provided by mixing 40 g of squalane and 15 cm³/g of the porous spherical disintegrative silica; the paste is impregnated between 2 mm thickness of 2 cm diameter parallel plates; and the dynamic viscoelasticity of the paste is measured at the conditions of 2 Hz angular frequency and 10 Pa to 10 kPa of increasing shear stress; the minimum value of [dlog storage modulus of elasticity]/[dlog shearing stress] is no less than about −10;

(b) the water-insoluble skin benefit agent having a solubility in water at less than about 0.1 g/l at 25° C. and having a molecular weight of no more than about 5,000, selected from the group consisting of liquid water-insoluble skin benefit agents, solid water-insoluble skin benefit agents which dissolve in liquid water-insoluble skin benefit agents, solid water-insoluble skin benefit agents which dissolve in emollients and/or volatile solvents, and mixtures thereof; and

(2) a suitable carrier.

By impregnating the water-insoluble skin benefit agents in the above mentioned porous spherical disintegrative silica, the water-insoluble skin benefit agent can be stably formulated in a variety of topical composition forms. Upon immediate application on the skin, the impregnated porous spherical disintegrative silica provides improved skin feel. When shear is applied to the skin for spreading the composition, the impregnated porous spherical disintegrative silica readily disintegrates, and the water-insoluble skin benefit agent becomes available for direct application on the skin.

These and other features, aspects, and advantages of the present invention will become evident to those skilled in the art from a reading of the present disclosure with the appended claims.

### DETAILED DESCRIPTION OF THE INVENTION

While the specification concludes with claims particularly pointing out and distinctly claiming the invention, it is believed that the present invention will be better understood from the following description.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

All ingredients such as actives and other ingredients useful herein may be categorized or described by their cosmetic and/or therapeutic benefit or their postulated mode of action. However, it is to be understood that the active and other ingredients useful herein can, in some instances, provide more than one cosmetic and/or therapeutic benefit or operate via more than one mode of action. Therefore, classifications herein are made for the sake of convenience and are not intended to limit an ingredient to the particularly stated application or applications listed. IMPREGNATED POROUS SPHERICAL DISINTEGRATIVE SILICA

The present invention comprises a porous spherical disintegrative silica impregnated with a water-insoluble skin benefit agent. By impregnating the water-insoluble skin benefit agent in the silica, the water-insoluble skin benefit agent has reduced reactivity, thereby causing less influence with the remainder of the topical composition during storage. Thus, the water-insoluble skin benefit agent can be stably formulated in a variety of topical composition forms. Further, upon immediate application to the skin, there is less influence in unfavorable skin feel, such as sticky and/or greasy feel, caused by the water-insoluble skin benefit agent. Due to the favorable skin feel of the porous spherical silica itself upon immediate application on the skin, the impregnated porous spherical disintegrative silica provides a fresh light feel to the skin. Thus, the present compositions containing the water-insoluble skin benefit agent impregnated in the porous spherical disintegrative silica has improved skin feel compared to compositions containing the water-insoluble skin benefit agent directly in the composition.

When shear is applied to the skin for spreading the composition, the impregnated porous spherical disintegrative silica readily disintegrates, and the water-insoluble skin benefit agent becomes available for direct application on the skin. The impregnated porous spherical disintegrative silica has certain physical properties which provide a structure rigid enough to be stable during regular mixing processes upon manufacture of the topical composition as well as during storage, yet brittle enough to disintegrate upon stress applied to the skin for spreading a topical composition. After disintegration, the water-insoluble skin benefit agent becomes available for direct application on the skin, and the disintegrated silica particles provide a moisturizing adhering skin feel compared to the condition before disintegration. After disintegration, the fragment silica particles fill the fine lines and pores on the skin, and fit well onto the skin. Due to the relatively low refraction index of silica itself, the fragment silica particles thus fit on the skin provides good coverage of fine lines and pores, while also providing a translucent appearance.

Porous Spherical Disintegrative Silica

The porous spherical disintegrative silica of the present invention for impregnating with a water-insoluble skin benefit agent are those described in Japanese Laid-open patent publication 2003-137760, incorporated herein by reference, having the following 3 physical properties.

(1) The porous spherical disintegrative silica of the present invention has a more or less spherical shape, and has an average volume particle size of from about 3 μm to about 20 μm, and a maximum particle size of no more than about 50 μm, preferably an average volume particle size of from about 4 μm to about 15 μm, and a maximum particle size of no more than about 30 μm, when measured by laser reflecting method. By having such shape and particle size, the porous spherical disintegrative silica of the present invention provides characteristics of spherical silica as commonly used in the cosmetic field. Namely, the porous spherical disintegrative silica of the present invention provides a fresh light feeling to the skin, without providing a feel of unevenness. The particle size is at least about 3 μm so that the sense of disintegration can be felt on the skin.

(2) The porous spherical disintegrative silica of the present invention has a pore volume of from about 1.5 cm³/g to about 3.0 cm³/g, preferably from about 1.7 cm³/g to about 2.5 cm³/g, when measured by nitrogen adsorption method. Such pore volume is necessary for providing volume to impregnate the water-insoluble skin benefit agents, while also providing a certain structural strength of the porous spherical disintegrative silica.

(3) The porous spherical disintegrative silica of the present invention has a minimum value of [dlog storage modulus of elasticity]/[dlog shearing stress] of no less than about −10, preferably no less than about −8, when measured with the following steps:

1. A paste is provided by mixing 40 g of squalane and 15 cm³/g of the porous spherical disintegrative silica

2. The paste is impregnated between 2 mm thickness of 2 cm diameter parallel plates.

3. The dynamic viscoelasticity of the paste is measured at the conditions of 2 Hz angular frequency and 10 Pa to 10 kPa of increasing shear stress.

By having such characteristics with regard to dynamic viscoelasticity, the porous spherical disintegrative silica is rigid enough to be stable during regular mixing processes upon manufacture of the topical composition as well as during storage, yet brittle enough to disintegrate upon stress applied to the skin for spreading a topical composition. Also, by having such characteristics with regard to dynamic viscoelasticity, the porous spherical disintegrative silica gradually disintegrates as more shear is applied, thus, capable of providing a fresh light feel to the skin upon immediate application on the skin, yet a moisturizing adhering skin feel later, as the disintegration proceeds. Such change in skin feel is particularly beneficial for providing the user with a signal that disintegration has happened, and the skin benefit agents have actually been released for application to the skin.

The porous spherical disintegrative silica suitable herein may be surface treated with silicone and other hydrophobic ingredients. Surface treatment may be provided before or after the skin benefit agent is impregnated.

Commercially available porous spherical disintegrative silica suitable for use herein include tradename SA-SB-705 Silica available from Miyoshi Kasei. SA-SB-705 Silica has an average particle size of about 5-6 μm, a pore volume of about 1.5-3.0 cm³/g, and is surface treated with 10% its weight of dimethicone.

Water-Insoluble Skin Benefit Agent

The water-insoluble skin benefit agent of the present invention to be impregnated into the porous spherical disintegrative silica have a solubility in water of less than about 0.1 g/l at 25° C. and having a molecular weight of no more than about 5,000, selected from the group consisting of liquid water-insoluble skin benefit agents, solid water-insoluble skin benefit agents which dissolve in liquid water-insoluble skin benefit agents, solid water-insoluble skin benefit agents which dissolve in emollients and/or volatile solvents, and mixtures thereof.

The water-insoluble skin benefit agents herein are active ingredients which provide a cosmetic and/or therapeutic effect to the area of topical application, which can be volatile or nonvolatile. The water-insoluble skin benefit agents are used in a safe and effective amount, by which is meant an amount high enough to deliver the desired skin benefit, but low enough to avoid unnecessary side effects at a reasonable benefit to risk ratio. The amount by weight of the water-insoluble skin benefit agent will vary with the specific agent, ability to penetrate through the skin, and other factors. The agents herein can be categorized by their cosmetic or therapeutic benefit or their postulated mode of action. However, it is understood by the artisan that, in some instances, the same agent may provide more than one cosmetic or therapeutic benefit, or operate via more than one mode of action. Water-insoluble skin benefit agents useful herein include skin lightening agents, skin soothing agents, skin repair agents, skin tightening agents, anti-acne agents, sebum inhibitors, anti-inflammatory agents, sensates and perfumes, UV absorbing agents, mixtures thereof, and others.

Skin lightening agents useful herein include ascorbic acid derivatives such as ascorbyl tetraisopalmitate (for example, VC-IP available from Nikko Chemical), ascorbyl palmitate (for example available from Roche Vitamins), ascorbyl dipalmitate (for example, NIKKOL CP available from Nikko Chemical); undecylenoyl phenyl alanine (for example, SEPIWHITE MSH available from Seppic); octadecenedioic acid (for example, ARLATONE DIOIC DCA available from Uniquema); oenothera biennis sead extract, and pyrus malus (apple) fruit extract, and mixtures thereof.

Skin soothing agents useful herein include pyridoxine dicaprylate (for example NIKKOL DK available from Nikko Chemical); pyridoxine dipalmitate (for example NIKKOL DP available from Nikko Chemical); vitamin B6 tetraisopalmitate, D-panthenyl ethyl alcohol; acetyl pantothenyl ethyl ether; benxoyl panthothenyl ethyl ether; lycopene (for example Lyc-O-Zone available from Roche Vitamins), riboflavin tetrabutylate, boswellia serrata extract (for example, SOOTHEX available from Quest International); borage oil, chamomile, evening primrose, tocopherol and its derivatives such as tocopherol acetate (for example, available from Eisai), and mixtures thereof.

Skin repair agents useful herein include retinol derivatives such as retinal, retinal, retinol palmitate, retinol acetate; and mixtures thereof.

Skin tightening agents useful herein include escin beta-sitosterol (for example, mixture with phospholipids available from Nikko Chemical); and cola acuminata seed extract.

Anti-acne agents useful herein include melalueca alternifolia (tea tree leaf oil) available as MELAFRESH EXFOL 100.

Sebum inhibitors useful herein include crataegus monogine flower extract available as HAWTHORN dry extract.

Antiinflammatory agents useful herein include algae extract, and artemisia vulgaris extract available as TRIPLE A COMPLEX.

Sensates and perfumes useful herein include camphor, thymol, limonene, menthol, menthyl lactate (for example FRESCOLATER ML available from Haarman & Reimer), eucalyptus, carboxamides, menthane ethers, menthane esters, γ-heptyl butyrolactone, ethyl β-methyl-β-phenyl glycidate, and natural and synthetic perfume components.

UV absorbing agents useful herein include 2-ethylhexyl-p-methoxycinnamate (commercially available as PARSOL MCX), butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropyl dibenzoylmethane, 3-benzylidene camphor, 3-(4-methylbenzylidene) camphor, and those by tradenames EUSOLEX 6300, OCTOCRYLENE, and PARSOL 1789.

Impregnation

Impregnation can be done by simply mixing the porous spherical disintegrative silica and the water-insoluble skin benefit agent in a suitable mixer under regular mixing conditions used for mixing pigments to homogeneous state. By such mixing, the water-insoluble skin benefit agent is absorbed into the porous spherical disintegrative silica via capillary action. When the water-insoluble skin benefit agent is solid at room temperature, the water-insoluble benefit agent is first dissolved into a carrier selected from the group consisting of a liquid water-insoluble skin benefit agent, an emollient, a volatile solvent, or mixtures thereof, and then the mixture is absorbed into the porous spherical disintegrative silica. The volatile solvent may be removed with the aid of heating. Emollients useful herein are the same as those described under the section “Oil Phase”. Volatile solvents useful herein are any that are capable of dissolving, yet inert, with the water-insoluble skin benefit agent. Volatile silicone oils can be used.

The amount of water-insoluble skin benefit agent to be impregnated into the porous spherical disintegrative silica depends on the pore volume of the porous spherical disintegrative silica, and the physical/chemical properties of the water-insoluble skin benefit agent. Typically, the impregnated porous spherical disintegrative silica comprises a maximum of about 80% water-insoluble skin benefit agent. Impregnation beyond or close to the maximum capacity of the porous spherical disintegrative silica may lead to releasing the impregnated or excess water-insoluble skin benefit agent to the composition during manufacturing or storage. Preferably, the impregnated porous spherical disintegrative silica comprises from about 20% to about 70% of the water-insoluble skin benefit agent.

While the water-insoluble skin benefit agent is at least incorporated in the present topical composition as being impregnated in the porous spherical disintegrative silica, a certain amount of water-insoluble skin benefit agent may also exist in the remainder of the composition. The amount allowed in the remainder of the composition will vary depending on the targeted skin feel and other characteristics of the product.

Suitable Carrier and Topical Compositions

The impregnated porous spherical disintegrative silica of the present invention can be incorporated in various topical compositions for delivering the water-insoluble skin benefit agent in a stable manner. Topical compositions which particularly benefit from the use of the impregnated porous spherical disintegrative silica of the present invention are those wherein skin feel is an important characteristic of the product. Improved skin feel may be recognized by the user as one or more of smooth spreadability, fresh light feeling, improved coverage, reduced sticky feel, and reduced greasy feel. Such topical compositions and their respective suitable carriers are listed hereinbelow.

Powder Cosmetic Compositions Comprising:

(a) from about 0.01% to about 30% of the impregnated porous spherical disintegrative silica;

(b) from about 74% to about 98% of a pigment; and

(c) from about 1% to about 25% of a binder.

Water-In-Oil Emulsion Cosmetic Compositions Comprising:

(a) from about 0.01% to about 30% of the impregnated porous spherical disintegrative silica;

(b) from about 20% to about 80% of a continuous oil phase;

(c) from about 1% to about 60% of a discontinuous aqueous phase;

(d) an emulsifier;

(e) a pigment; and

(f) optionally a wax for providing the composition to be solid at room temperature.

Oil-In-Water Emulsion Cosmetic Compositions Comprising:

(a) from about 0.01% to about 30% of the impregnated porous spherical disintegrative silica;

(b) from about 20% to about 80% of a continuous aqueous phase;

(c) from about 1% to about 60% of a discontinuous oil phase;

(d) an emulsifier; and

(e) optionally a pigment.

Lipophilic Cosmetic Compositions Comprising:

(a) from about 0.01% to about 30% of the impregnated porous spherical disintegrative silica;

(b) from about 1% to about 20% of thickener; and

(c) a volatile solvent selected from the group consisting of hydrocarbon oil and silicone oil.

Oil Phase

The topical compositions of the present invention may contain an oil phase. The oil phase may be made of emollients, silicone oils, UV absorbing agents as mentioned above, and mixtures thereof. When UV absorbing agents are included in the oil phase, the type and amount are carefully selected in view of the overall skin feel of the topical composition.

Emollients useful herein are various grades of mineral oils. Mineral oils are liquid mixtures of hydrocarbons that are obtained from petroleum. Specific examples of suitable hydrocarbons include paraffin oil, mineral oil, dodecane, isododecane, hexadecane, isohexadecane, eicosene, isoeicosene, tridecane, tetradecane, polybutene, polyisobutene, hydrogenated polydecene, and mixtures thereof. Also useful as emollients herein are, for example, tridecyl isononanoate, isostearyl isostearate, isocetyl isosteatrate, isopropyl isostearate, octyldodecyl myristate, ethyl linoleate, isopropyl linoleate, isodecyl isonoanoate, cetyl octanoate, isononyl isononanoate, diisopropyl myristate, isocetyl myristate, isotridecyl myristate, isopropyl myristate, isostearyl palmitate, isocetyl palmitate, isodecyl palmitate, isopropyl palmitate, octyl palmitate, diethylhexyl 2,6-naphthalate, octyldodecyl neopentaoate, butyloctyl salicylate, phytosteryl isostearate, caprylic/capric acid triglyceride, glyceryl tri-2-ethylhexanoate, neopentyl glycol di(2-ethyl hexanoate), diisopropyl dimerate, avocado oil, camellia oil, turtle oil, macadamia nut oil, corn oil, mink oil, olive oil, rapeseed oil, eggyolk oil, sesame oil, persic oil, wheat germ oil, pasanqua oil, castor oil, linseed oil, safflower oil, cotton seed oil, perillic oil, soybean oil, peanut oil, tea seed oil, kaya oil, rice bran oil, china paulownia oil, Japanese paulownia oil, jojoba oil, rice germ oil, glycerol trioctanate, glycerol triisopalmiatate, trimethylolpropane triisostearate, isopropyl myristate, glycerol tri-2-ethylhexanoate, pentaerythritol tetra-2-ethylhexanoate, lanolin, liquid lanolin, liquid paraffin, squalane, vaseline, cholesteryl derivatives such as cholesteryl 12-hydroxystearate, cholesteryl macadamiate, cholesteryl stearate, cetyl ricinolate, phytantriol, and mixtures thereof. Commercially available oils include, for example, tridecyl isononanoate with tradename CRODAMOL TN available from Croda, HEXALAN available from Nisshin Seiyu, cholesteryl 12-hydroxystearate with tradename SALACOS HS available from Nisshin Oil Mills, Ltd., cholesterol with tradename CHOLESTEROL JPK available from Nippon Fine Chemical, cholesteryl lanolate with tradename YOFOC CLE-S, cholesteryl oleate with tradename YOFCO LC-CO-D, lanolin with tradename SUPER LIQUID LANOLIN, and mixtures thereof.and cholesteryl macadamiate with tradename YOFCO MAC available from Nippon Fine Chemical Co., Ltd.

Silicone oils are useful herein. Particularly useful are those which have low viscosity but are not too volatile, preferably those having a viscosity of less than about 60 mPas and a volatility as such that not more than 35% of the silicone oil evaporates after standing at 150° C. at normal pressure for 24 hours. Such silicone oils are believed to provide a fresh and light feel when the composition is applied to the skin.

Silicone oils useful herein also include polyalkyl or polyaryl siloxanes with the following structure (I) 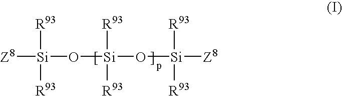
wherein R⁹³ is alkyl or aryl, and p is an integer from about 7 to about 100. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the skin, is compatible with the other components of the composition, and is chemically stable under normal use and storage conditions. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. Polyalkylaryl siloxane fluids can also be used and include, for example, polymethylphenylsiloxanes. Commercially available herein include methylphenyl polysiloxane with tradenames KF56 available from ShinEtsu Chemical Co., Ltd., SF1075 METHYL PHENYL FLUID available from the General Electric Company, 556 COSMETIC GRADE FLUID available from Dow Corning, polydimethylsiloxane having less than 50 n-Pas with tradenames SH200 available from Dow Coming and the VISCASIL and SF96 series available from the General Electric Company, and mixtures of Dimethicone and Dimethiconol with tradenames Q2-1403 fluid and Q2-1503 fluid available from Dow Corning.
Emulsifier

The topical compositions of the present invention may contain an emulsifier. The emulsifier is selected depending on the other components of the composition of the present invention, and provides the desired emulsification or dispersion characteristics. Suitable emulsifiers have an HLB value of from about 4 to about 14. Emulsifiers having an HLB value outside of these ranges can be used in combination with other emulsifiers to achieve an effective weighted average HLB for the combination that falls within these ranges.

Useful silicone emulsifiers include dimethicone copolyols. These materials are polydimethyl siloxanes which have been modified to include polyether side chains such as polyethylene oxide chains, polypropylene oxide chains, mixtures of these chains, and polyether chains containing moieties derived from both ethylene oxide and propylene oxide. Other examples include alkyl-modified dimethicone copolyols, i.e., compounds which contain C2-C30 pendant side chains. Still other useful dimethicone copolyols include materials having various cationic, anionic, amphoteric, and zwitterionic pendant moieties. Examples of commercially available dimethicone copolyols useful herein sold by Dow Corning Corporation are Dow Corning® 190, 193, Q2-5220, 2501 Wax, 2-5324 fluid, and 3225C (this later material being sold as a mixture with cyclomethicone). Cetyl dimethicone copolyol is commercially available as a mixture with polyglyceryl-4 isostearate (and) hexyl laurate and is sold under the tradename ABIL® WE-09 (available from Goldschmidt). Cetyl dimethicone copolyol is also commercially available as a mixture with hexyl laurate (and) polyglyceryl-3 oleate (and) cetyl dimethicone and is sold under the tradename ABIL® WS-08 (also available from Goldschmidt). Other nonlimiting examples of dimethicone copolyols also include lauryl dimethicone copolyol, dimethicone copolyol acetate, diemethicone copolyol adipate, dimethicone copolyolamine, dimethicone copolyol behenate, dimethicone copolyol butyl ether, dimethicone copolyol hydroxy stearate, dimethicone copolyol isostearate, dimethicone copolyol laurate, dimethicone copolyol methyl ether, dimethicone copolyol phosphate, and dimethicone copolyol stearate. See International Cosmetic Ingredient Dictionary, Fifth Edition, 1993.

Among the non-silicone-containing emulsifiers useful herein are various non-ionic and anionic emulsifying agents such as sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, soaps, and mixtures thereof. Other suitable emulsifiers are described, for example, in McCutcheon's, Detergents and Emulsifiers, North American Edition (1986), published by Allured Publishing Corporation; U.S. Pat. No. 5,011,681 to Ciotti et al., issued Apr. 30, 1991; U.S. Pat. No. 4,421,769 to Dixon et al., issued Dec. 20, 1983; and U.S. Pat. No. 3,755,560 to Dickert et al., issued Aug. 28, 1973.

Nonlimiting examples of these non-silicon-containing emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, PEG-100 stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, steareth-20, ceteareth-20, PPG-2 methyl glucose ether distearate, ceteth-10, diethanolamine cetyl phosphate, glyceryl stearate, PEG-100 stearate, and mixtures thereof.

Among the nonionic surfactants that are useful herein are those that can be broadly defined as condensation products of long chain alcohols, e.g. C8-30 alcohols, with sugar or starch polymers, i.e., glycosides. These compounds can be represented by the formula (S)ₙ—O—R wherein S is a sugar moiety such as glucose, fructose, mannose, and galactose; n is an integer of from about 1 to about 1000, and R is a C8-30 alkyl group. Examples of long chain alcohols from which the alkyl group can be derived include decyl alcohol, cetyl alcohol, stearyl alcohol, lauryl alcohol, myristyl alcohol, oleyl alcohol, and the like. Preferred examples of these surfactants include those wherein S is a glucose moiety, R is a C8-20 alkyl group, and n is an integer of from about 1 to about 9. Commercially available examples of these surfactants include decyl polyglucoside (available as APG 325 CS from Henkel) and lauryl polyglucoside (available as APG 600 CS and 625 CS from Henkel).

Other useful nonionic surfactants include the condensation products of alkylene oxides with fatty acids (i.e. alkylene oxide esters of fatty acids). These materials have the general formula RCO(X)ₙOH wherein R is a C10-30 alkyl group, X is —OCH₂CH₂— (i.e. derived from ethylene glycol or oxide) or —OCH₂CHCH₃— (i.e. derived from propylene glycol or oxide), and n is an integer from about 6 to about 200. Other nonionic surfactants are the condensation products of alkylene oxides with 2 moles of fatty acids (i.e. alkylene oxide diesters of fatty acids). These materials have the general formula RCO(X)ₙOOCR wherein R is a C10-30 alkyl group, X is —OCH₂CH₂—(i.e. derived from ethylene glycol or oxide) or —OCH₂CHCH₃—(i.e. derived from propylene glycol or oxide), and n is an integer from about 6 to about 100. Other nonionic surfactants are the condensation products of alkylene oxides with fatty alcohols (i.e. alkylene oxide ethers of fatty alcohols). These materials have the general formula R(X)ₙOR′ wherein R is a C10-30 alkyl group, X is —OCH₂CH₂—(i.e. derived from ethylene glycol or oxide) or —OCH₂CHCH₃— (i.e. derived from propylene glycol or oxide), and n is an integer from about 6 to about 100 and R′ is H or a C10-30 alkyl group. Still other nonionic surfactants are the condensation products of alkylene oxides with both fatty acids and fatty alcohols [i.e. wherein the polyalkylene oxide portion is esterified on one end with a fatty acid and etherified (i.e. connected via an ether linkage) on the other end with a fatty alcohol]. These materials have the general formula RCO(X)ₙOR′ wherein R and R′ are C10-30 alkyl groups, X is —OCH₂CH₂ (i.e. derived from ethylene glycol or oxide) or —OCH₂CHCH₃— (derived from propylene glycol or oxide), and n is an integer from about 6 to about 100. Nonlimiting examples of these alkylene oxide derived nonionic surfactants include ceteth-6, ceteth-10, ceteth-12, ceteareth-6, ceteareth-10, ceteareth-12, steareth-6, steareth-10, steareth-12, steareth-21, PEG-6 stearate, PEG-10 stearate, PEG-100 stearate, PEG-12 stearate, PEG-20 glyceryl stearate, PEG-80 glyceryl tallowate, PEG-10 glyceryl stearate, PEG-30 glyceryl cocoate, PEG-80 glyceryl cocoate, PEG-200 glyceryl tallowate, PEG-8 dilaurate, PEG-10 distearate, and mixtures thereof.

Still other useful nonionic surfactants include polyhydroxy fatty acid amide surfactants corresponding to the structural formula: 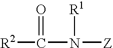
wherein: R¹ is H, C₁-C₄ alkyl, 2-hydroxyethyl, 2-hydroxy- propyl, preferably C₁-C₄ alkyl, more preferably methyl or ethyl, most preferably methyl; R² is C₅-C₃₁ alkyl or alkenyl, preferably C₇-C₁₉ alkyl or alkenyl, more preferably C₉-C₁₇ alkyl or alkenyl, most preferably C₁₁-C₁₅ alkyl or alkenyl; and Z is a polhydroxyhydrocarbyl moiety having a linear hydrocarbyl chain with a least 3 hydroxyls directly connected to the chain, or an alkoxylated derivative (preferably ethoxylated or propoxylated) thereof. Z preferably is a sugar moiety selected from the group consisting of glucose, fructose, maltose, lactose, galactose, mannose, xylose, and mixtures thereof. An especially preferred surfactant corresponding to the above structure is coconut alkyl N-methyl glucoside amide (i.e., wherein the R²CO— moiety is derived from coconut oil fatty acids). Processes for making compositions containing polyhydroxy fatty acid amides are disclosed, for example, in G.B. Patent Specification 809,060, published Feb. 18, 1959, by Thomas Hedley & Co., Ltd.; U.S. Pat. No. 2,965,576, to E. R. Wilson, issued Dec. 20, 1960; U.S. Pat. No. 2,703,798, to A. M. Schwartz, issued Mar. 8, 1955; and U.S. Pat. No. 1,985,424, to Piggott, issued Dec. 25, 1934; which are incorporated herein by reference in their entirety.

Preferred among the nonionic surfactants are those selected from the group consisting of steareth-21, ceteareth-20, ceteareth-12, sucrose cocoate, steareth-100, PEG-100 stearate, and mixtures thereof.

Other nonionic surfactants suitable for use herein include sugar esters and polyesters, alkoxylated sugar esters and polyesters, C1-C30 fatty acid esters of C1-C30 fatty alcohols, alkoxylated derivatives of C1-C30 fatty acid esters of C1-C30 fatty alcohols, alkoxylated ethers of C1-C30 fatty alcohols, polyglyceryl esters of C1-C30 fatty acids, C1-C30 esters of polyols, C1-C30 ethers of polyols, alkyl phosphates, polyoxyalkylene fatty ether phosphates, fatty acid amides, acyl lactylates, and mixtures thereof. Nonlimiting examples of these emulsifiers include: polyethylene glycol 20 sorbitan monolaurate (Polysorbate 20), polyethylene glycol 5 soya sterol, Steareth-20, Ceteareth-20, PPG-2 methyl glucose ether distearate, Ceteth-10, Polysorbate 80, cetyl phosphate, potassium cetyl phosphate, diethanolamine cetyl phosphate, Polysorbate 60, glyceryl stearate, polyoxyethylene 20 sorbitan trioleate (Polysorbate 85), sorbitan monolaurate, polyoxyethylene 4 lauryl ether sodium stearate, polyglyceryl-4 isostearate, hexyl laurate, PPG-2 methyl glucose ether distearate, PEG-100 stearate, and mixtures thereof.

Another emulsifier useful herein are fatty acid ester blends based on a mixture of sorbitan or sorbitol fatty acid ester and sucrose fatty acid ester, the fatty acid in each instance being preferably C₈-C₂₄, more preferably C₁₀-C₂₀. The preferred fatty acid ester emulsifier is a blend of sorbitan or sorbitol C₁₆-C₂₀ fatty acid ester with sucrose C₁₀-C₁₆ fatty acid ester, especially sorbitan stearate and sucrose cocoate. This is commercially available from ICI under the trade name Arlatone 2121.

Binder

The topical compositions of the present invention may contain a binder. The term binder as used herein means the component used in powder foundation products for keeping the product together. The amount and type of binder is selected depending on the desired characteristic of the product, for example, product form such as loose powder or compact powder, coverage, adhesion to the skin, and various skin feel.

Components useful as a binder include oil phase material and emulsifiers as mentioned above.

Thickener

The thickeners useful herein are selected from the group consisting of fatty compounds, wax, gelling agents, inorganic thickeners, silicone elastomers, water soluble polymers, water in-soluble polymers, and mixtures thereof. The amount and type of thickeners are selected according to the desired viscosity and characteristics of the product.

Fatty Compounds

Fatty compounds useful herein include stearic acid, palmitic acid, stearyl alcohol, cetyl alcohol, behenyl alcohol, stearic acid, palmitic acid, the polyethylene glycol ether of stearyl alcohol or cetyl alcohol having an average of about 1 to about 5 ethylene oxide units, and mixtures thereof. Preferred fatty compounds of the present invention are selected from stearyl alcohol, cetyl alcohol, behenyl alcohol, the polyethylene glycol ether of stearyl alcohol having an average of about 2 ethylene oxide units (steareth-2), the polyethylene glycol ether of cetyl alcohol having an average of about 2 ethylene oxide units, and mixtures thereof.

Wax

The waxes useful herein are paraffin wax, microcrystalline wax, ozokerite wax, ceresin wax, carnauba wax, candellila wax, silicone wax, eicosanyl behenate, and mixtures thereof. A mixture of waxes is preferably used. Commercially available waxes useful herein include: Candelilla wax NC-1630 available from Noda wax, Ozokerite wax SP-1021 available from Strahl & Pitsh, Eicosanyl behenate available from Cas Chemical, and Cetyl Dimethicone with tradename ABIL WAX 9801 available from Goldschmidt.

Gelling Agents

The gelling agents useful as thickeners of the present invention include esters and amides of fatty acid gellants, hydroxy acids, hydroxy fatty acids, other amide gellants, and crystalline gellants.

N-acyl amino acid amides useful herein are prepared from glutamic acid, lysine, glutamine, aspartic acid and mixtures thereof. Particularly preferred are n-acyl glutamic acid amides corresponding to the following formula:
R²—NH—CO—(CH₂)₂—CH—(NH—CO—R¹)—CO—NH—R²
wherein R¹ is an aliphatic hydrocarbon radical having from about 12 to about 22 carbon atoms, and R² is an aliphatic hydrocarbon radical having from about 4 to about 12 carbon atoms. Non-limiting examples of these include n-lauroyl-L-glutamic acid dibutyl amide, n-stearoyl-L-glutamic acid diheptyl amide, and mixtures thereof. Most preferred is n-lauroyl-L-glutamic acid dibutyl amide, also referred to as dibutyl lauroyl glutamide. This material is commercial available with tradename Gelling agent GP-1 available from Ajinomoto.

Other gelling agents suitable for use in the compositions include 12-hydroxystearic acid, esters of 12-hydroxystearic acid, amides of 12-hydroxystearic acid and combinations thereof. These preferred gellants include those which correspond to the following formula:
R¹—CO—(CH₂)₁₀—CH—(OH)—(CH₂)₅—CH₃
wherein R¹ is R² or NR²R³; and R² and R³ are hydrogen, or an alkyl, aryl, or arylalkyl radical which is branched linear or cyclic and has from about 1 to about 22 carbon atoms; preferably, from about 1 to about 18 carbon atoms. R² and R³ may be either the same or different; however, at least one is preferably a hydrogen atom. Preferred among these gellants are those selected from the group consisting of 12-hydroxystearic acid, 12-hydroxystearic acid methyl ester, 12-hydroxystearic acid ethyl ester, 12-hydroxystearic acid stearyl ester, 12-hydroxystearic acid benzyl ester, 12-hydroxystearic acid amide, isopropyl amide of 12-hydroxystearic acid, butyl amide of 12-hydroxystearic acid, benzyl amide of 12-hydroxystearic acid, phenyl amide of 12-hydroxystearic acid, t-butyl amide of 12-hydroxystearic acid, cyclohexyl amide of 12-hydroxystearic acid, l-adamantyl amide of 12-hydroxystearic acid, 2-adamantyl amide of 12-hydroxystearic acid, diisopropyl amide of 12-hydroxystearic acid, and mixtures thereof; even more preferably, 12-hydroxystearic acid, isopropyl amide of 12-hydroxystearic acid, and combinations thereof. Most preferred is 12-hydroxystearic acid.

Suitable amide gellants include disubstituted or branched monoamide gellants, monosubstituted or branched diamide gellants, triamide gellants, and combinations thereof, excluding the n-acyl amino acid derivatives selected from the group consisting of n-acyl amino acid amides, n-acyl amino acid esters prepared from glutamic acid, lysine, glutamine, apartic acid, and combinations thereof, and which are specifically disclosed in U.S. Pat. No. 5,429,816.

Alkyl amides or di- and tri-basic carboxylic acids or anhydrides suitable for use in the composition include alkyl amides of citric acid, tricarballylic acid, aconitic acid, nitrilotriacetic acid, succinic acid and itaconic acid such as 1,2,3-propane tributylamide, 2-hydroxy-1,2,3-propane tributylamide, 1-propene-1,2,3-triotylamide, N,N′,N″-tri(acetodecylamide)amine, 2-dodecyl-N,N′-dihexylsuccinamide, and 2 dodecyl-N,N′-dibutylsuccinamide. Preferred are alkyl amides of di-carboxylic acids such as di-amides of alkyl succinic acids, alkenyl succinic acids, alkyl succinic anhydrides and alkenyl succinic anhydrides, more preferably 2-dodecyl-N,N′-dibutylsuccinamide.

Inorganic Thickeners

Inorganic thickeners useful herein include hectorite, bentonite, montmorillonite, and bentone clays which have been modified to be compatible with oil. Preferably, the modification is quatemization with an ammonium compound. Preferable inorganic thickeners include quaternary ammonium modified hectorite. Commercially available oil swelling clay materials include benzyldimethyl stearyl ammonium hectorite with tradename Bentone 38 available from Elementis.

Silicone Elastomers

Suitable for use herein are silicone elastomers which can be emulsifying or non-emulsifying crosslinked siloxane elastomers or mixtures thereof. The term “non-emulsifying,” as used herein, defines crosslinked organopolysiloxane elastomers from which polyoxyalkylene units are absent. The term “emulsifying,” as used herein, means crosslinked organopolysiloxane elastomers having at least one polyoxyalkylene (e.g., polyoxyethylene or polyoxypropylene) unit. Non-emulsifying elastomers useful in the present invention are formed via crosslinking organohydroenpolysiloxanes with an alpha, omega-diene. Emulsifying elastomers herein include polyoxyalkylene modified elastomers formed via crosslinking from organohydrogenpolysiloxanes with polyoxyalkylene dienes or organohydrogenpolysiloxanes containing at least one polyether group crosslinked with an alpha, omega-diene. Emulsifying crosslinked organopolysiloxane elastomer can notably be chosen from the crosslinked polymers described in U.S. Pat. No. 5,412,004 (issued May 2, 1995); U.S. Pat. No. 5,837,793 (issued Nov. 17, 1998); and U.S. Pat. No. 5,811,487 (issued Sep. 22, 1998). In addition, an emulsifying elastomer comprised of dimethicone copolyol crosspolymer (and dimethicone) is available from Shin Etsu under the tradename KSG-21.

Non-emulsifying elastomers are dimethicone/vinyl dimethicone crosspolymers. Such dimethicone/vinyl dimethicone crosspolymers are supplied by a variety of suppliers including Dow Corning (DC 9040 and DC 9041), General Electric (SFE 839), Shin Etsu (KSG-15, 16, 18 [dimethicone/phenyl vinyl dimethicone crosspolymer]), and Grant Industries (GRANSIL™ line of elastomers). Cross-linked organopolysiloxane elastomers useful in the present invention and processes for making them are further described in U.S. Pat. No. 4,970,252 to Sakuta, et al., issued Nov. 13, 1990; U.S. Pat. No. 5,760,116 to Kilgour, et al., issued Jun. 2, 1998; U.S. Pat. No. 5,654,362 to Schulz, Jr., et al. issued Aug. 5, 1997. Additional crosslinked organopolysiloxane elastomers useful in the present invention are disclosed in Japanese Patent Application JP 61-18708, assigned to Pola Kasei Kogyo K K. Commercially available elastomers preferred for use herein are Dow Corning's 9040 silicone elastomer blend, Shin Etsu's KSG-21, and mixtures thereof

Water-Soluble Polymers

Water-soluble polymers are useful herein. Such polymers include carboxylic acid polymers, polyacrylamide polymers, polysaccharides, gums, film forming polymers, and mixtures thereof.

Carboxylic acid polymers include crosslinked compounds containing one or more monomers derived from acrylic acid, substituted acrylic acids, and salts and esters of these acrylic acids and the substituted acrylic acids, wherein the crosslinking agent contains two or more carbon-carbon double bonds and is derived from a polyhydric alcohol. Polymers useful in the present invention are more fully described in U.S. Pat. No. 5,087,445, to Haffey et al, issued Feb. 11, 1992; U.S. Pat. No. 4,509,949, to Huang et al, issued Apr. 5, 1985; U.S. Pat. No. 2,798,053, to Brown, issued Jul. 2, 1957; and in CTFA International Cosmetic Ingredient Dictionary, Fourth Edition, 1991, pp. 12 and 80. Examples of commercially available carboxylic acid polymers useful herein include the carbomers, which are homopolymers of acrylic acid crosslinked with allyl ethers of sucrose or pentaerytritol. The carbomers are available as the Carbopol® 900 series from B.F. Goodrich (e.g., Carbopol® 954). In addition, other suitable carboxylic acid polymeric agents include copolymers of C₁₀₋₃₀ alkyl acrylates with one or more monomers of acrylic acid, methacrylic acid, or one of their short chain (i.e., C₁₋₄ alcohol) esters, wherein the crosslinking agent is an allyl ether of sucrose or pentaerytritol. These copolymers are known as acrylates/C₁₀₋₃₀ alkyl acrylate crosspolymers and are commercially available as Carbopol® 1342, Carbopol® 1382, Pemulen TR-1, and Pemulen TR-2, from B.F. Goodrich. In other words, examples of carboxylic acid polymer thickeners useful herein are those selected from carbomers, acrylates/C₁₀-C₃₀ alkyl acrylate crosspolymers, and mixtures thereof.

Polyacrylamide polymers, especially nonionic polyacrylamide polymers including substituted branched or unbranched polymers are useful herein. More preferred among these polyacrylamide polymers is the nonionic polymer given the CTFA designation polyacrylamide and isoparaffin and laureth-7, available under the Tradename Sepigel 305 from Seppic Corporation (Fairfield, N.J.). Other polyacrylamide polymers useful herein include multi-block copolymers of acrylamides and substituted acrylamides with acrylic acids and substituted acrylic acids. Commercially available examples of these multi-block copolymers include Hypan SR150H, SS500V, SS500W, SSSA100H, from Lipo Chemicals, Inc., (Patterson, N.J.).

A wide variety of polysaccharides are useful herein. “Polysaccharides” refer to gelling agents which contain a backbone of repeating sugar (i.e., carbohydrate) units. Nonlimiting examples of polysaccharide gelling agents include those selected from cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroxyalkylated (preferably hydroxyethylated or hydroxypropylated) to form a hydroxyalkylated cellulose which is then further modified with a C₁₀-C₃₀ straight chain or branched chain alkyl group through an ether linkage. Typically these polymers are ethers of C₁₀-C₃₀ straight or branched chain alcohols with hydroxyalkylcelluloses. Examples of alkyl groups useful herein include those selected from stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof. Preferred among the alkyl hydroxyalkyl cellulose ethers is the material given the CTFA designation cetyl hydroxyethylcellulose, which is the ether of cetyl alcohol and hydroxyethylcellulose. This material is sold under the tradename Natrosol® CS Plus from Aqualon Corporation (Wilmington, Del.).

Water-soluble, film-forming polymers useful herein are formed from monomers selected from the group consisting of olefin oxides, vinyl pyrrolidone, vinyl esters, vinyl alcohols, vinyl cyanides, oxazilines, carboxylic acids and esters and mixtures thereof. Preferred vinyl pyrrolidone polymers are selected from the group consisting of polyvinylpyrrolidone, vinyl acetate/vinyl pyrrolidone copolymer and mixtures thereof. Preferred polyvinyl esters are selected form the group consisting of vinyl acetate/crotonic acid copolymer, vinyl acetate/crotonic acid/vinyl neodecanoate copolymer and mixtures thereof. Preferred vinyl alcohol polymers are selected from the group consisting of vinyl alcohol/vinyl acetate, vinyl alcohol/poly(alkyleneoxy)acrylate, vinyl alcohol/vinyl acetate/poly-(alkyleneoxy)acrylate and mixtures thereof. Preferred olefin oxides are selected from the group consisting of polyethylene oxide, polypropylene oxide and mixtures thereof. Preferred polycarboxylic acids and their esters are selected from the group consisting of acrylates, acrylates/octylacrylamide copolymers and mixtures thereof. The preferred oxazilines is polyoxazilines.

Specific water-soluble, film-forming polymers useful in the present invention include, but are not necessarily limited to Polyox WSR (polyethyleneoxide polymers) from Union Carbide; Airvol (polyvinylalcohol copolymer) from Air Products and Chemicals, preferably all commercially available grades like Airvol 103, Airvol 325, Airvol 540, Airvol 523S; Vinex from Air Products and Chemicals, preferably all commercially available grades such as Vinex 1003, Vinex 2034, Vinex 2144, Vinex 2019; PEOX.(polyethyloxazoline) from Polymer Chemistry Innovations; PVP K Series (polyvinylpyrrolidone) from International Specialty Products; Luviskol K Series (polyvinylpyrrolidone) from BASF; PVP/VA (vinyl acetate/vinyl pyrrolidone copolymer) from International Specialty Products, preferably grades W-735 and S-630; and Gantrez (copolymers of methyl vinyl ether/maleic anhydride) from International Specialty Products; Polymer EX33-9 available from Interpolymer (acrylate copolymer); Carboset Series (acrylate copolymer) from BF Goodrich; Resyn Series (vinyl acetate/crotonate copolymers) from National Starch and Chemical Corporation; Versatyl and Dermacryl Series (acrylate/octylacrylamide copolymers) from National Starch and Chemical Corporation.

Water-Insoluble Polymers

Water-insoluble polymers are useful herein. These polymers comprise monomers selected from the group consisting of aromatic vinyls, dienes, vinyl cyanides, vinyl halides, vinylidene halides, vinyl esters, olefins and their isomers, vinyl pyrrolidone, unsaturated carboxylic acids, alkyl esters of unsaturated carboxylic acids, hydroxy derivatives of alkyl esters of unsaturated carboxylic acids, amides of unsaturated carboxylic acids, amine derivatives of unsaturated carboxylic acids, glycidyl derivatives of alkyl esters of unsaturated carboxylic acids, olefinic diamines and isomers, aromatic diamines, terephthaloyl halides, olefinic polyols and mixtures thereof. Preferred monomers are selected from the group consisting of aromatic vinyls, dienes, vinyl esters, olefins and their isomers, unsaturated carboxylic acids, alkyl esters of unsaturated carboxylic acids, hydroxy derivatives of alkyl esters of unsaturated carboxylic acids, arnides of unsaturated carboxylic acids and mixtures thereof. Most preferred monomers are selected from the group consisting of aromatic vinyls, dienes, vinyl esters, alkyl esters of unsaturated carboxylic acids, hydroxy derivatives of alkyl esters of unsaturated carboxylic acids and mixtures thereof. The polymerization process for making said polymeric material of the present invention is well known in the art. Such processes are disclosed in Kirk Othmer, Encyclopedia of Chemical Technology, Volume 14, “Latex Technology” 3rd Ed. 1981; incorporated herein by reference.

Commercially available water-insoluble polymers useful herein include Syntran Series (of latexes) from Interpolymer Corporation, for example Syntran 5170, Syntran EX33-1, Syntran EX30-1, and Syntran 5130 (acrylates copolymers formulated with added ammonia, propylene glycol, preservative and surfactant) and Syntran 5002 (styrene/acrylates/methacrylate copolymer formulated with added ammonia, propylene glycol, preservative and surfactant); the Primal Series (acrylic latexes) from Rohm & Hass; Appretan V (styrene/acrylic ester copolymer latexes) from Hoechst; Vinac (polyvinylacetate latex) from Air Products; UCAR latex resin 130 (polyvinylacetate latex) from Union Carbide; Rhodopas A Series (polyvinylacetate latexes) from Rhone Poulenc; Appretan MB, EM, TV (vinyl acetate/ethylene copolymer latexes) from Hoechst; 200 Series (styrene/butadiene copolymer latexes) from Dow Chemical; Rhodopas SB Series (styrene/butadiene copolymer latexes) from Rhone Poulenc; Witcobond (polyurethane latexes) from Witco; Hycar Series (butadiene/acrylonitrile copolymer latexes) from Goodrich; Chemigum Series (butadiene/acrylonitrile copolymer latexes) from Goodyear; and Neo Cryl (styrene/acrylates/acrylonitrile copolymer latex) from ICI Resins.

Aqueous Phase

The topical composition of the present invention may comprise an aqueous phase. Water for use in the aqueous phase may be deionized water, or water from natural sources. Various water-soluble ingredients may be included in the aqueous phase, such as water-soluble humectants and water-soluble active ingredients.

Pigment

The topical composition of the present invention may comprise a pigment. Pigments useful herein include those that provide color or change tone, and also those that provide a certain skin feel.

The base powders useful herein include clay mineral powders such as talc, mica, sericite, silica, magnesium silicate, synthetic fluorphlogopite, calcium silicate, aluminum silicate, bentonite and montomorilonite. The coloring powders useful herein include pearl pigments such as alumina, barium sulfate, calcium secondary phosphate, calcium carbonate, zirconium oxide, zinc oxide, hydroxy apatite, iron oxide, iron titate, ultramarine blue, Prussian blue, chromium oxide, chromium hydroxide, cobalt oxide, cobalt titanate, titanium oxide coated mica; organic powders such as polyester, polyethylene, polystyrene, methyl metharylate resin, cellulose, 12-nylon, 6-nylon, styrene-acrylic acid copolymers, poly proprylene, vinyl chloride polymer, tetrafluoroethylene polymer, boron nitride, fish scale guanine, laked tar color dyes, and laked natural color dyes. Such base powders, titanium dioxide, and coloring powders may be treated with a hydrophobical treatment agent, including: silicone such as Methicone, Dimethicone and perfluoroalkylsilane; fluorine such as diethanolamine salts of perfluoroalkyl phosphate, fatty material such as stearic acid; metal soap such as aluminium dimyristate; aluminium hydrogenated tallow glutamate, hydrogenated lecithin, lauroyl lysine, aluminium salt of perfluoroalkyl phosphate, and mixtures thereof.

Spherical powders other than the impregnated porous spherical disintegrative silica may also be used. Unlimited examples of materials useful for making the spherical powders are; polyacrylates, silicates, sulfates, alumina, metal dioxides, carbonates, celluloses, polyalkylenes, vinyl acetates, polystyrenes, polyamides, acrylic acid ethers, silicones, and mixtures and complexes thereof. Specifically, materials useful herein include polyacrylates such as methyl methacrylate copolymer and nylon, cross linked polymethyl methacrylate; silicates such as calcium silicate, magnesium silicate, barium silicate, aluminium silicate and silica beads; alumina; metal dioxides such as titanium dioxide and aluminium hydroxide; carbonates such as calcium carbonate, magnesium carbonate; celluloses; polyalkylenes such as polyethylene, and polypropylene; vinyl acetates; polystyrenes; polyamides; acrylic acid ethers such as acrylic acid methyl ether and acrylic acid ethyl ether; polyvinyl pyrrolidones; and silicones such as polyorganosilsesquioxane resin and solid silicone elastomers. Highly preferred materials are polymethyl methacylate.

In one embodiment, polyorganosilsesquioxane resin and solid silicone elastomers may be used for enhancing the effect of hiding skin pores.

Commercially available spherical powders highly useful herein include methyl methacylate copolymer with tradename GANZ PEARL series available from Ganz Chemical Co., Ltd., and SYLYSIA series available from Fuji Sylysia Chemical, Nylon-12 with tradename NYLON POWDER series available from Toray Dow Corning, vinyl dimethicone/methicone silsesquioxane crosspolymer with tradenames KSP series available from ShinEtsu Chemical Co., Ltd., Tokyo Japan, and hardened polyorgano siloxane elastomers with tradenames TREFIL series available from Toray Dow Coming.

Additional Components

The composition of the present invention may include other additional components which may be selected by the artisan according to the desired characteristics of the final product and which are suitable for rendering the composition more cosmetically or aesthetically acceptable or to provide them with additional usage benefits. Such additional components generally are used individually at levels of no more than about 5% by weight of the composition.

Other components which can be formulated into the compositions of the present invention are; preservatives such as benzyl alcohol, methyl paraben, propyl paraben, sodium dehydro acetate, niacinamide, imidazolidinyl area, and EDTA and its salts, and perfumes.

### EXAMPLES

The following examples further describe and demonstrate the preferred embodiments within the scope of the present invention. The examples are given solely for the purpose of illustration, and are not to be construed as limitations of the present invention since many variations thereof are possible without departing from its spirit and scope.

### Examples 1-4

The following are examples of impregnated porous disintegrative silica components useful for the topical compositions of the present invention, which are made by the impregnation process as described above.

Example 1: 50% ascorbyl tetraisopalmitate, 45% porous disintegrative silica, and 5% dimethicone (surface treatment)

Example 2: 50% vitamin B6 tetraisopalmitate, 45% porous disintegrative silica, and 5% dimethicone (surface treatment)

Example 3: 25% ethylhexyl methoxycinnamate, 25% undecylenoyl phenylalanine, 45% porous disintegrative silica, and 5% dimethicone (surface treatment)

Example 4: 25% ethylhexyl methoxycinnamate, 25% octadecenedioic acid, 45% porous disintegrative silica, and 5% dimethicone (surface treatment)

Example 5: 50% ethylhexyl methoxycinnamate, 45% porous disintegrative silica, and 5% dimethicone (surface treatment)

Example 6: 20% menthyl lactate, 72% porous disintegrative silica, and 8% dimethicone (surface treatment)

Definition of Materials:

Porous disintegrative silica surface treated with dimethicone: SA-SB-705 Silica available from Miyoshi Kasei

Ascorbyl tertaisopalmitate: VC-IP available from Nikko Chemical

Vitamin B6 tetraisopalmitate: available from Nikko Chemical

Undecylenoyl Phenylalanine: SEPIWHITE MSH available from SEPPIC

Octadecenedioic Acid: Arlatone Dioic DCA is available from Uniquema

Ethylhexyl Methoxycinnamate: Parsol MCX available from Roche.

Menthyl Lactate: Frescolate ML available from Haarman&Reimer K.K.

### Examples 11-35

The following are topical compositions of the present invention containing the impregnated porous disintegrative silica components of Examples 1-6.

Composition: Tables 1-3 list the components used in each composition.

TABLE 1

| No. | Components | Ex. 11 | Ex. 12 | Ex. 13 | Ex. 14 |
|---|---|---|---|---|---|
| 1 | Example 1 | | | 6 | |
| 2 | Example 2 | 1 | | | |
| 3 | Example 3 | | 4 | | |
| 4 | Example 4 | | | | 8 |
| 5 | Methyl methacrylate | 2 | | 2 | 2 |
| | crosspolymer | | | | |
| | 20 μm (*1) | | | | |
| 6 | Methyl methacrylate | 6 | | 6 | 6 |
| | crosspolymer 8 μm (*2) | | | | |
| 7 | Methyl methacrylate | 12 | 10 | 12 | 12 |
| | crosspolymer | | | | |
| | 6 μm (*3) | | | | |
| 8 | Sericite coated | | 30 | | |
| | with Methicone (*5) | | | | |
| 9 | Titanium Dioxide | 12 | | 12 | 12 |
| | coated | | | | |
| | with Methicone (*8) | | | | |
| 10 | Sericite coated with | 35 | | 30 | 30 |
| | C9-15 fluoroalcohol | | | | |
| | phosphates and | | | | |
| | triethoxycaprylylsilane | | | | |
| | (*6) | | | | |
| 11 | Talc coated with | | | | |
| | Methicone (*4) | | q.s to | | |
| | | | 100% | | |
| 12 | Talc coated with C9-15 | q.s to | | q.s to | q.s to |
| | fluoroalcohol phosphates | 100% | | 100% | 100% |
| | and | | | | |
| | triethoxycaprylylsilane (*7) | | | | |
| 13 | Methylparaben | 0.3 | 0.3 | 0.3 | 0.3 |
| 14 | Propylparaben | 0.1 | 0.1 | 0.1 | 0.1 |
| 15 | Iron Oxide coated | | | | |
| | with Methicone (*9) | 2.5 | 2.5 | 2.5 | 2.5 |
| 16 | Dimethicone (*10) | 5.0 | 7.0 | 6.5 | 5.0 |
| 17 | Ethylhexyl | | | | |
| | Methoxycinnamate (*11) | 4 | 4 | 4 | |

TABLE 2

| No. | Components | Ex. 21 | Ex. 22 | Ex. 23 | Ex. 24 |
|---|---|---|---|---|---|
| 1 | Example 2 | 2 | | | |
| 2 | Example 5 | 2 | | | 2 |
| 3 | Example 1 | | 2 | | |
| 4 | Example 3 | | | 4 | |
| 5 | Polymethylsilesquioxane | 3 | | | |
| | (*15) | | | | |
| 6 | Isohexadecane (*16) | 2 | 3.0 | 3.0 | |
| 7 | Isopropyl | | 2 | 2 | |
| | Isostearate (*17) | | | | |
| 8 | Carnauba wax (*18) | | | | 3 |
| 9 | Paraffin Wax (*19) | | | | 2 |
| 10 | Dimethicone & | 2.0 | | | |
| | Dimethiconol-1 (*23) | | | | |
| 11 | Dimethicone & | | 2 | 2 | |
| | Dimethiconol-2 (*24) | | | | |
| 12 | Glycerin, conc (*13) | | 7.0 | 7.0 | 1 |
| 13 | Polyvinyl Alcohol (*14) | | | | 2 |
| 14 | Triethanolamine (*25) | 0.8 | | | 2 |
| 15 | Cetyl Alcohol (*26) | | 0.5 | 0.5 | |
| 16 | Stearyl Alcohol (*27) | | 0.6 | 0.6 | |
| 17 | Behenyl Alcohol (*28) | | 0.4 | 0.4 | |
| 18 | Cetearyl Alcohol & | | 0.1 | 0.1 | |
| | Cetearyl Glucoside (*29) | | | | |
| 19 | PEG-100 Stearate (*31) | | 0.1 | 0.1 | |
| 20 | Stearic Acid (*30) | | 0.1 | 0.1 | 4 |
| 21 | Glyceryl Stearate | | | | 10 |
| 22 | Lecithin (*20) | | | | 1 |
| 23 | Quaternium-18 | | | | 6 |
| | Hectorite (*21) | | | | |
| 24 | Acrylates/C10-30 | 0.35 | | | |
| | Alkyl Acrylate | | | | |
| | Crosspolymer (*32) | | | | |
| 25 | Polyacrylamide & Water& | | 0.2 | 0.2 | |
| | C13-14 Isoparaffin& | | | | |
| | Laureth-7 (*33) | | | | |
| 26 | Acrylate Copolymer (*34) | | | | 15 |
| 27 | Propylene Carbonate (*22) | | | | 2 |
| 28 | Black Iron Oxides (*35) | | | | 7 |
| 29 | Benzyl Alcohol | 0.2 | 0.25 | 0.25 | |
| 30 | Methylparaben | 0.12 | 0.12 | 0.12 | 0.3 |
| 31 | Propylparaben | | 0.07 | 0.07 | 0.1 |
| 32 | Ethylparaben | | 0.05 | 0.05 | |
| 33 | Disodium EDTA | 0.1 | 0.1 | 0.1 | |
| 34 | Perfume | 0.02 | 0.03 | 0.03 | |

| | | |
|---|---|---|
| 35 | Water | q.s to100% |

TABLE 3

| No. | Components | Ex.31 | Ex.32 | Ex.33 | Ex.34 | Ex.35 |
|---|---|---|---|---|---|---|
| 1 | Example 1 | 20 | | 1 | 5 | |
| 2 | Example 6 | | | | 5 | |
| 3 | Example 2 | | | 3 | | |
| 4 | Example 5 | | | | | 2 |
| 5 | Example 4 | | 7 | | | |
| 6 | Cyclopentasiloxane | 5 | 12 | 3 | 3 | |
| | (and) PEG/PEG- | | | | | |
| | 18/18 Dimethicone (*39) | | | | | |
| 7 | Cetyl Dimethicone (*40) | | | 3 | | |
| 8 | Cyclomethicone& | | | | 50 | |
| | Dimethicone Cross | | | | | |
| | Polymer (*37) | | | | | |
| 9 | Dimethicone Copolyol | | | 3 | 3 | |
| | Crosspolmer (*38) | | | | | |
| 10 | Laureth-7 (*43) | | | 1 | | |
| 11 | Sorbitan | 2 | 2 | | | |
| | Monoisostearate (*41) | | | | | |
| 12 | Quaternium-18 | | | | | 10 |
| | Hectorite (*21) | | | | | |
| 13 | Pentaerythrityl | | | | | 5 |
| | Hydrogenated | | | | | |
| | Rosinate | | | | | |
| | (*42) | | | | | |
| 14 | Candelilla wax (*44) | 1.5 | 3 | | | |
| 15 | Ceresin (*45) | 1.5 | | | | |
| 16 | Microcrystalline Wax (*46) | | 2 | | | |
| 17 | Carnauba wax (*18) | | | | | 2 |
| 18 | Trihydroxystearin (*47) | | | 1 | | 2 |
| 19 | Arachidyl Behenate (*48) | | | 1 | | |
| 20 | Talc coated with | 2 | | 2 | | |
| | Methicone (*4) | | | | | |
| 21 | Mica coated with | 12 | | 2 | 3 | |
| | Methicone (*36) | | | | | |
| 22 | Titanium Dioxide coated | 9 | 2.5 | 10 | 5 | |
| | with Methicone (*8) | | | | | |
| 23 | Iron Oxide coated with | 3.0 | | 2.5 | 2.0 | 10 |
| | Methicone (*9) | | | | | |
| 24 | Water | 10 | 20 | 45 | 10 | |
| 25 | Propylene Carbonate (*22) | | | | | 4 |
| 26 | Butylene Glycol (*12) | 10 | 5 | | | |
| 27 | Methylparaben | 0.2 | 0.2 | 0.2 | 0.2 | 0.3 |
| 28 | Propylparaben | 0.1 | 0.25 | 0.1 | 0.1 | 0.1 |
| 29 | Disodium EDTA | 0.1 | 0.1 | 0.1 | 0.1 | |
| 30 | Cyclopentasiloxane (*49) | q.s to 100% | | | | |
| 31 | C₁₀₋₁₁ Isoparaffin (*50) | | | | | q.s to |
| | | | | | | 100% |

Definitions of Components

1 Methyl methacrylate crosspolymer: GANZ PEARL GMX-2001 available from GANZ CHEMICAL CO., LTD.

2 Methyl methacrylate crosspolymer: GANZ PEARL GMX-801 available from GANZ CHEMICAL CO., LTD.

3 Methyl methacrylate crosspolymer: GANZ PEARL GMX-601 available from GANZ CHEMICAL CO., LTD.

4 Talc coated with Methicone: SI TALC available from MIYOSHI KASEI, INC.

5 Sericite coated with Methicone: SI SERICITE available from MIYOSHI KASEI, INC.

6 Sericite coated with C9-15 fluoroalcohol phosphates and triethoxycaprylylsilane: FOTS SERICITE FSE available from DAITO KASEI KOUGYOU CO., LTD.

7 Talc coated with C9-15 fluoroalcohol phosphates and triethoxycaprylylsilane: FOTS TALC JA-46R available from DAITO KASEI KOUGYOU CO., LTD

8 Titanium Dioxide coated with Methicone: SI TITANIUM DIOXIDE IS available from TOSHIKI PIGMENT CO., LTD.

9 Iron Oxide coated with Methicone: IRON OXIDE series available from DAITO KASEI KOUGYOU CO., LTD.

10 Dimethicone: SH200 available from Dow Corning

11 Ethylhexyl Methoxycinnamate: PARSOL MCX available from ROCHE VITAMINS JAPAN K.K.

12 Butylene Glycol: 1,3-Butylene Glycol available from Matsumoto Trading Co.

13 Glycerin available from Shin Nihon Rika

14 Polyvinyl Alcohol: Vinex 2019 available from Texas Polymer

15 Polymethylsilesquioxane: Tospearl 145A available from Toshiba-GE Silicone

16 Isohexadecane: Permethyl 101A available from Presperse

17 Isopropyl Isostearate: NIKKOL EPIS available from NIKKO Chemicals

18 Carnauba Wax: Purified Carnauba wax No.1 available from Noda Co. Ltd.

19 Paraffin Wax: PT-0602 available from Astor Wax Corporation

20 Lecithin: Centrex F available from Central Soya

21 Quaternium-18 Hectorite: Bentone 38 available from Elementis

22 Propylene Carbonate: Propylene Carbonate available from Nisso Petrochemical Industries

23 Dimethicone & Dimethiconol-1: Dow Corning Q2-1403 available from Dow Corning

24 Dimethicone & Dimethiconol-2: Dow Corning Q2-1503 fluid available from Dow Coming

25 Triethanolamine: Triethanolamine-S available from Nihon Shokubai Kagaku Kogyo

26 Cetyl Alcohol: Cetyl Alcohol available from Shin Nihon Rika

27 Stearyl Alcohol: Stearyl Alcohol available from Shin Nihon Rika

28 Behenyl Alcohol: NIKKOL Behenyl Alcohol available from NIKKO Chemicals

29 Cetearyl Alcohol & Cetearyl Glucoside: Emulgade PL-68/50 available from Cognis

30 Stearic Acid: Hystrene 5016 available from Witco Chemical

31 PEG-100 Stearate: PEG-100 Stearate available from Uniqema

32 Acrylates/C10-30 Alkyl Acrylate Crosspolymer: Pemulen TR-2 availble from B.F.Goodrich

33 Polyacrylamide & Water& C13-14 Isoparaffin& Laureth-7: Sepigel 305 available from SEPPIC

34 Acrylates Copolymer: Polymer EX33-9 available from Interpolymer

35 Black Iron Oxide: Sicovit Black 80E 172

36 Mica coated with Methicone: SI MICA available from MIYOSHI KASEI, INC.

37 Cyclomethicone&Dimethicone Crosspolymer: DC9040 available from Dow Corning

38 Dimethicone Copolyol Crosspolymer: KSG-21 available from ShinEtsu Silicone

39 Cyclopentasiloxane (and) PEG/PEG-18/18 Dimethicone: Silicone DC5225C available from Dow Corning

40 Cetyl Dimethicone: ABIL WAX 9801 available from Goldschmidt

41 Sorbitan Monoisostearate: CRILL6 available from Croda JAPAN

42 Pentaerythrityl Hydrogenated Rosinate: Rikatack F105 available from Rika Finetech K.K.

43 Laureth-7: POE-7 Lauryl Ether available from Sanyo Kasei Kogyo.

44 Candelilla wax: CANDELILLA WAX NC-1630 available from Noda Wax.

45 Ceresin: OZOKERITE WAX available from Ina Trade

46 Microcrystalline Wax: MICRYSTALLINE WAX available from Iwase Cosfa Co. Ltd.

47 Trihydroxystearin: Thixcin R available from Elementis

48 Arachidyl Behenate: Waxenol 822 available from ALZO

49 Cyclopentasiloxane: Silicone DC245 available from Dow Corning

50 C10-C11 Isoparaffin: Amso mineral Sprits available from Ashland Chemical

Method of Preparation

The compositions of Examples 11-35 are prepared as follows.

### Examples 11-14

1) Component numbers 1-15 are mixed with a suitable mixer until homogeneous to make a pigment mixture. The pigment mixture is pulverized using a pulverizer.

2) Component numbers 16 and 17 are added into the pigment mixture and mixed by a mixer until homogeneous. The obtained mixture is further pulverized using a pulverizer.

3) The obtained composition is pressed in a tray and set into a compact.

### Example 21

1) Component numbers of 1-2, 5-6, 10, 24, 29-30, and 34 are mixed until homogenous with a suitable mixer equipped with a homogenizer.

2) Separately, component numbers 14 and 33 are mixed in 35 until dissolved.

3) The product of 2) is added into the product of 1) and mixed until uniform using a suitable mixer equipped with a propeller blade.

4) The obtained emulsion is poured into a suitable container.

### Examples 22-23

1) Component numbers 12 and 35 are poured into a suitable mixing tank and heated to about 70-80° C.

2) Component numbers 6-7, 11, 15-20, and 29-33 are added into the product of 1) and mixed until homogenous with a mixer equipped with a homogenizer to effect emulsification.

3) The product of 2) is cooled to about 60° C., and component number 25 is added under mixing.

4) The product of 3) is further cooled to about 40-50° C., and component numbers 2 or 4 and 34 are added, and then milled using suitable mill until uniform.

5) The obtained emulsion is poured into a suitable container.

### Example 24

1) Component numbers 8-9, 14, 20-23, and 27-28 are added into a suitable mixing tank and heated to about 90° C. while mixing using homogenizer.

2) Separately, component number 35 is heated in a suitable mixing tank to about 90° C. and component numbers 12-13 are added while mixing with a mixer equipped with a propeller blade.

3) The product of 2) is added into product of 1) while mixing using homogenizer until uniform.

4) The product of 3) is cooled to about 60-65° C., and component numbers 2, 26, and 30-31 are added while mixing using homogenizer. 5) The product of 4) is further cooled to room temperature while mixing using homogenizer.

6) The obtained composition is poured into a suitable container.

### Examples 31-32

1) Component numbers 6, 11, 20-23, and 30 are added in a sealed tank and mixed at room temperature using a homogenizer.

2) The product of 1) is heated to about 80-85° C. and component numbers 14-16 are added and mixed until completely dissolved.

3) Separately, component numbers 26-29 are dissolved in component number 24 by heating to about 75° C.

4) The product of step 3) is addd to the product of step 2) for emulsification while mixing using homogenizer.

5) The emulsion of step 4) is cooled to about 60-70° C., and component numbers 1 or 5 are added into the emulsion.

6) The obtained composition, while still fluid, is filled in an air tight container and allowed to cool to room temperature to solidify with or without using a cooling unit. The air-tight container is typically in a form of a package for compact.

### Example 33

1) Component numbers 6-7 and 30 are mixed in a sealed tank and heated to about 85-90° C. and then component numbers 9, and 19-23 are added while mixing using homogenizer.

2) The product of 1) is cooled to about 60° C., and component number 18 is added.

3) The product of 2) is further cooled to about 40° C., and component numbers 10 and 27-29 are added while mixing and continuing to mix until the batch is room temperature.

4) Component numbers 1, 3, and 24 are added to the product of 3) at room temperature while mixing using homogenizer. The emulsion is mixed until uniform.

5) The obtained composition is poured into a suitable container.

### Example 34

1) Component numbers 1-2, 6, 9, 21-23 and 30 are mixed in a suitable mixer using homogenizer.

2) Separately, component numbers of 27-29 are dissolved into 24 at about 70-80° C.

3) The products of 1) and 2) are mixed to effect emulsification, and mixed until uniform.

4) Component number 8 is added to the product of 3) and mixed using homogenizer.

5) The obtained composition is poured into a suitable container.

### Example 35

1) Component numbers 12-13, 17-18, 23, 25 and 31 are added in a sealed mixing tank and heated to about 90° C. while mixing using homogenizer.

2) The product of 1) is cooled to about 60-65° C., and component numbers 4, and 27-28 are added while mixing using homogenizer.

3) The product of 2) is cooled to room temperature while mixing using homogenizer.

4) The obtained composition is poured into a suitable container.

Product Form and Usage

Examples 11-14 are embodiments of compositions suitable for use as pressed powder foundation products. When applied on the facial skin, they provide many advantages. For example, they can provide balanced benefits in terms of shine control, transfer resistance, smooth spreadability when applying on the skin, and improved adhesion on the skin. When chronically used, they can provide skin lightening benefit. Example 11 further provides skin soothing benefit when chronically used. Examples 12 and 14 provide UV protection benefit.

Examples 21-23 are embodiments of oil-in-water emulsion phase type compositions suitable for use as lotions. When applied on the facial skin, they provide many advantages. For example, they can provide smooth feel without sticky and greasy feel when applying on the skin, while proving moisturization to the skin, and UV protection benefit. When chronically used, they provide skin lightening benefit, and Example 21 further provides skin soothing benefit.

Example 24 is an embodiment of a oil-in-water emulsion phase type composition suitable as mascara. When applied on eyelashes, they provide many advantages. For example, they can provide improved body and better separation without sticky and greasy feel when applying on the eyelashes, and UV protection benefit.

Examples 31-32 are embodiments of water-in-oil emulsion phase type compositions suitable for use as solid foundation products. When applied on the facial skin, they provide many advantages. For example, they can provide balanced benefits in terms of spreadability when applying on the skin, improved adhesion on the skin, improved coverage, and fresh light feel on the skin, and provide effective UV protection effect. When used chronically, they provide skin lightening benefit.

Examples 33-34 are embodiments of water-in-oil emulsion phase type compositions suitable for use as liquid foundation products. When applied on the facial skin, they provide many advantages. For example, they can provide balanced benefits in terms of superior spreadability when applying on the skin, improved adhesion on the skin, improved coverage, and fresh light feel on the skin. When used chronically, they provide skin lightening benefit, and Example 33 further provides skin soothing benefit.

Example 35 is an embodiment of a composition suitable for use as mascara. When applied on the eyelashes, they provide many advantages. For example, they can provide long lasting in curl, improved body and better separation without sticky and greasy feel when applying on the eyelashes, and UV protection benefit.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A topical composition comprising:
(1) a porous spherical disintegrative silica impregnated with a water-insoluble skin benefit agent, wherein:
(a) the porous spherical disintegrative silica has an average volume particle size of from about 3 μm to about 20 μm, a maximum particle size of no more than 50 μm, and a pore volume of from about 1.5 cm³/g to about 3.0 cm³/g; wherein when a paste is provided by mixing 40 g of squalane and 15 cm³/g of the porous spherical disintegrative silica; the paste is impregnated between 2 mm thickness of 2 cm diameter parallel plates; and the dynamic viscoelasticity of the paste is measured at the conditions of 2 Hz angular frequency and 10 Pa to 10 kPa of increasing shear stress; the minimum value of [dlog storage modulus of elasticity]/[dlog shearing stress] is no less than about −10;
(b) the water-insoluble skin benefit agent having a solubility in water at less than about 0.1 g/l at 25° C. and having a molecular weight of no more than about 5,000, selected from the group consisting of liquid water-insoluble skin benefit agents, solid water-insoluble skin benefit agents which dissolve in liquid water-insoluble skin benefit agents, solid water-insoluble skin benefit agents which dissolve in emollients and/or volatile solvents, and mixtures thereof; and
(2) a suitable carrier.

2. The topical composition of claim 1 wherein the skin benefit agent is selected from the group consisting of skin lightening agents, skin soothing agents, skin repair agents, and mixtures thereof.

3. The topical composition of claim 1 wherein the skin benefit agent comprises ultraviolet absorbing agents.

4. The topical composition of claim 1 wherein the skin benefit agent comprises sensates and perfumes.

5. The topical composition of claim 1 wherein the composition is a powder cosmetic composition comprising:
(a) from about 0.01% to about 30% of the impregnated porous spherical disintegrative silica;
(b) from about 74% to about 98% of a pigment; and
(c) from about 1% to about 25% of a binder.

6. The topical composition of claim 1 wherein the composition is a water-in-oil emulsion cosmetic composition comprising:
(a) from about 0.01% to about 30% of the impregnated porous spherical disintegrative silica;
(b) from about 20% to about 80% of a continuous oil phase;
(c) from about 1% to about 60% of a discontinuous aqueous phase;
(d) an emulsifier; and
(e) a pigment.

7. The water-in-oil cosmetic composition of claim 6 further comprising a sufficient amount of a wax for providing the composition to be solid at room temperature.

8. The topical composition of claim 1 wherein the composition is an oil-in-water emulsion cosmetic composition comprising:
(a) from about 0.01% to about 30% of the impregnated porous spherical disintegrative silica;
(b) from about 20% to about 80% of a continuous aqueous phase;
(c) from about 1% to about 60% of a discontinuous oil phase; and
(d) an emulsifier.

9. The oil-in-water cosmetic composition of claim 8 further comprising a pigment.

10. The topical composition of claim 1 wherein the composition is a lipophilic cosmetic composition comprising:
(a) from about 0.01% to about 30% of the impregnated porous spherical disintegrative silica;
(b) from about 1% to about 20% of thickener; and
(c) a volatile solvent selected from the group consisting of hydrocarbon oil and silicone oil.
